SEQUENCE LISTING (4 sequences)

SEQ ID NO: 1 - DNA, 14 bp, Artificial Sequence
  Description of Artificial Sequence Synthetic probe
accccgtccccatg

SEQ ID NO: 2 - DNA, 14 bp, Artificial Sequence
  Description of Artificial Sequence Synthetic probe
accccgtcctcatg

SEQ ID NO: 3 - DNA, 23 bp, Artificial Sequence
  Description of Artificial Sequence Synthetic probe
ggccactgactgatttgtgtgta

SEQ ID NO: 4 - DNA, 24 bp, Artificial Sequence
  Description of Artificial Sequence Synthetic probe
aagaaatggaggcaataggttttg

(19) United States
(12) Patent Application Publication
(10) Pub. No.: US 2003/0148288 A1
(43) Pub. Date: Aug. 7, 2003

(54) Colorimetric genetic test for clinically significant TNF polymorphism and methods of use thereof

(76) Inventor: Yi-Wei Tang, Brentwood, TN (US)

Correspondence Address: Douglas W. Schelling, Ph.D., Waddey & Patterson, Bank of America Plaza, 414 Union Street, Suite 2020, Nashville, TN, 37219, US

(21) Appl. No.: 10/062,248

(22) Filed: Feb. 1, 2002

(51) Int. Cl. C12Q 1/68; C12P 19/34

(52) U.S. Cl. 435/6; 435/91.2

(57) ABSTRACT

The present invention discloses a method and diagnostic kit used to identify polymorphisms. The method makes use of a minor groove binder and shorter hybridization probes so that polymorphisms, including polymorphisms of only a single nucleotide, may be detected. The present invention additionally discloses methods using hybridization conditions which increase the sensitivity of each individual nucleotide within the region being assayed. The invention additionally includes a diagnostic kit which supplies the necessary materials as well as instructions so that the method disclosed herein may be practiced in a time efficient manner.

### BACKGROUND OF THE INVENTION

Septic shock is a medical condition that has a mortality rate of 50%. Medical researches have concluded that tumor necrosis factor alpha (TNFα) plays a key role in the inflammatory response. Although TNFα plays a role in the inflammatory response, it is clear that an excessive amount of TNFα, while pathogenic microorganisms or their toxins are present in the blood in or in the tissues, may result in a worsened condition of a patient and consequently a worsened outcome for the patient.

Polymorphisms of the promoter region of the TNFα gene have been identified. More specifically, one of the polymorphisms results in a single nucleotide substitution of adenosine for guanine at position −308. More specifically, guanine at position −308 is termed TNF1 (wild type) and the substitution of adenosine at position −308 is termed TNF2. (Wilson, et al., Proc. Natl. Acad. Sci., April 1997, 94:3195-3199). Scientific research indicates that polymorphism TNF2 causes a significant increase in the transcriptional activity of the promoter for the TNFα gene compared to TNF1.

The gold standard for determining host polymorphisms has been the direct sequencing of a gene. However, cheaper and user-friendly methods are always in demand. Examples of such methods are restriction enzyme analysis, allele-specific hybridization, and denaturing gradient gel electrophoresis. Additional methods include gel electrophoresis or Southern hybridization, both of which are laborious in time consuming methods. Consequently, rapid and inexpensive methods which yield test results on the same day of specimen submission are in high demand. Such methods would not require the use of toxic chemical agents or electrophoresis apparatus. Such systems would take a short period of time to use such that a result is reached during the same day on which the sample arrives.

Individual genetic variations will become the basis for establishing health risks and customizing therapies. An example of such customization is shown for specific polymorphisms in the gene encoding TNFα, a critical pro-inflammatory cytokine. That polymorphism dictates the clinical outcome in patients with serious infection such as malaria, leishmaniasis, and septic shock.

Septic shock results from gram negative or gram positive bacteria and is the leading cause of death in intensive care units in this country, accounting for almost 100,000 deaths per year. Accordingly, each patient admitted to the intensive care unit that is at risk for septic shock would have use for a polymorphism kit which would permit rapid risk stratification in at risk patients either prior to acquiring septic shock or early in the course of the disease. A similar calorimetric microtiter plate assay is described by Tang, et al. (Tang, et al., Diag. Microbiol. Infect. Dis. (1999) 34:333-337).

The TNFα gene, which is 3.6 Kb in size, is located within the class III region of the major histocompatibility complex between HLA-B and HLA-DR on chromosome six. TNFα expression is tightly controlled at both the transcriptional and posttranscriptional levels. Several polymorphisms have been identified in the TNFα promoter region which would be expected to alter the amount of TNFα that is transcribed in response to any given stimulus. For example, it has been shown that a mutation of G to A at position −308 of the TNFα promoter results in a five-fold increase in transcriptional activity. (Wilson AG, et al., Proc. Natl. Acad. Sci. (1997) 94:3195-3199). The presence of the less common allele, termed TNF2, is associated with increased susceptibility to cerebral malaria, and septic shock. (McGuire, et al., Nature (1994) 371:508-510; Cabrera, et al., J. Exp. Med. (1995) 182:1259-1264; McGuire, et al., J. Infect. Dis. (1997) 176:530-532; Hohler, et al., Clin. Exp. Immunol. (1998) 111:579-580; Mira, et al., J.A.M.A. (1999) 282:561-568). It has been shown that individuals with the TNF2 allele are more severely struck when they have a disease. (Chagani, et al., Am. J. Respir. Crit. Care Med. (1999) 160:278-282; Hull, et al., Thorax (1998) 53:1018-1021; Sakao, et al., Am. J. Respir. Crit. Care Med. (2001) 163:420-422).

Knowledge that the response to drug therapy is dependent upon the genotype of the patient is nothing new. During World War II, it was noted that the risk of hemolytic anemia during antimalarial therapy was increased in individuals with G6PD deficiency. (Beutler, et al., J. Lab. Clin. Med. (1955) 45:40-50).

Polymorphisms in the TNFα locus have been shown to predict disease severity in many disease states, including, chronic pulmonary inflammatory diseases and multiple types of arthritis, including rheumatoid arthritis. Accordingly it is useful to characterize the TNFα polymorphism genotype in patients having and not having a significant clinical response to TNFα blockade therapy in order to correlate the clinical response to the TNFα genotype profile. Additionally, genotype profiling may be used to determine individual risk to certain diseases or syndromes. Knowing such genotypes would allow the application of preventative measures, if any. Having knowledge of the genotype of an individual would additionally allow a physician to employ appropriate therapy regimen based upon the known genotype. TNFα polymorphisms have been shown to determine severity of disease in such diseases as, asthma, cystic fibrosis, systemic lupus erythematosus, and rheumatoid arthritis, where chronic inflammation is a major pathogenic factor.

### SUMMARY OF THE INVENTION

The present invention discloses a method and kit that is used to determine the genotype of the patient in order to identify patients that will be responsive to drug therapy so that expensive and/or difficult to obtain drugs can be received by those patients who are most likely to respond. Doing so will save a considerable amount of health care cost.

Accordingly, one aspect of the invention is to limit the unnecessary exposure of individuals, which have been identified as non-responsive to a drug based upon their genotype, to non-responsive drugs.

Still another aspect of the invention is to determine individual risk to certain diseases or syndromes.

Another aspect of the invention is that methods described herein are user-friendly as they do not require toxic chemical agents or any other laborious to use apparatus. The method provides the additional benefit that it takes less than eight hours and may be adapted for automation.

Still another aspect of the invention is to provide a method of identifying genetic information so that such genetic information can be provided to a physician in order for the physician to prepare therapeutic regimen based upon the genetic information of the individual.

Another aspect of the invention is to provide a method of identifying a nucleotide variation in sequence in a nucleic acid. In certain embodiments, such method comprises providing a biological sample having a nucleic acid, contacting the nucleic acid with a primer under conditions for nucleic acid amplification, wherein the primer amplifies the nucleic acid in order to produce an amplification product, wherein the nucleic acid is amplified by polymerase chain reaction, contacting a first aliquot of the amplification product with a first solution having a first biotin-labeled probe, having a minor groove binder, so that an amplification product-probe complex is formed, and detecting the amplification product-probe complex.

Another aspect of the invention is to identify a nucleotide variation in sequence of a nucleic acid by incubating a first solution on a first streptavidin coated surface so that a first biotin-labeled probe is fixed to the coated surface. In certain embodiments a second aliquot of an amplification product may be contacted with a second solution which has a second biotin-labeled probe, such that the second biotin-labeled probe hybridizes to the amplification product. In certain embodiments it may be advantageous to fix the position of the second biotin-labeled probe by incubating the second solution on a second streptavidin coated surface. In certain embodiments the presence of digoxigenin-11-dUTP of the amplification product is detected on the second streptavidin coated surface by using peroxidase conjugated anti-digoxigenin-11-dUTP antibody. In alternate embodiments the second biotin-labeled probe further comprises a nucleic acid sequence having a length from about 7 nucleotides to about 15 nucleotides that hybridizes to the sequence for TNF2. Alternatively, it is possible for the second biotin-labeled probe to further comprise SEQ ID NO 2. In certain embodiments the first biotin-labeled probe may further comprise a nucleic acid sequence having a length of about 7 nucleotides to about 15 nucleotides that hybridizes to the sequence for TNF1. Alternatively, the first biotin-labeled probe may further comprise SEQ ID NO 1.

Still another aspect of the invention is to provide a method for identifying a genetic predisposition in a mammal. In certain embodiments, the method comprises providing a biological sample having genomic DNA from the mammal, amplifying the tumor necrosis factor alpha genomic DNA, or portion thereof, in order to produce an amplification product, providing a hybridization solution so that a probe hybridizes to the amplification product, hybridizing the probe to the amplification product so that an amplification product-probe complex is formed, immobilizing the probe, and determining the presence of the amplification product-probe complex wherein the presence of the amplification product-probe complex identifies a single nucleotide polymorphism in the TNFα genomic DNA. In certain embodiments the probe used to hybridize to the amplification product is SEQ ID NO 1. In other embodiments, the probe used to hybridize to the amplification product is SEQ ID NO 2. In alternate embodiments a first portion of the amplification product is mixed with a first hybridization solution.

Another aspect of the invention is to provide a method of identifying a genetic predisposition of an animal wherein a first hybridization solution, and a probe, which has a minor groove binder, the probe having a sequence of either SEQ ID NO 1 or SEQ ID NO 2, or a complementary sequence thereof is used. In certain embodiments the biological sample which is used to provide the genomic DNA of TNFα is a blood, plasma, or other bodily fluid sample. In alternate embodiments, the method is practiced upon mammals diseased with rheumatoid arthritis, septic shock, multiple sclerosis, Hepatitis B, Hepatitis C, chronic obstructive pulmonary disease, and certain cancers.

Still another aspect of the present invention is to provide a diagnostic kit for detecting single nucleotide polymorphisms in mammals. In certain embodiments, such a diagnostic kit comprises a forward PCR primer, a reverse PCR primer, a minor groove binding component, a first probe, a second probe, and a set of instructions for use. In certain embodiments, the length of the forward PCR primer and the reverse PCR primer is about 15 to about 100 nucleotides in length. Within said embodiment, the forward PCR primer and reverse PCR primer further comprise nucleic acid sequence that is complementary to the nucleic acid sequence of tumor necrosis factor alpha or to the promoter of tumor necrosis factor alpha. In other embodiments, the forward PCR primer and reverse PCR primer further comprise nucleic acid sequence that is complementary to the nucleic acid sequence of human tumor necrosis factor alpha or to the promoter of human tumor necrosis factor alpha. In alternate embodiments, the first probe comprises SEQ ID NO 1. In other embodiments a second probe comprises SEQ ID NO 2. In alternate embodiments the diagnostic kit additionally comprises digoxigenin-11-dUTP. In other embodiments the diagnostic kit also includes a streptavidin coated surface which may be used to immobilize the biotin-labeled probes. The diagnostic kit may additionally include a peroxidase-conjugated anti-digoxigenin-11-dUTP antibody.

### DESCRIPTION OF THE PREFERRED EMBODIMENTS

Disclosed herein is a method and kit for identifying polymorphisms to TNFα. This disclosed method allows the identification of the presence of a polymorphism to TNFα in order for such information to be employed while using other therapeutic methods to treat any illnesses related to the presence of the polymorphism. The polymorphism kit disclosed by the inventors includes instructions and all necessary materials in order for the method to be employed.

As used herein, the term “tumor necrosis factor alpha” (TNFα) means the TNFα of a human subject. The TNFα gene is a 3.6 Kb gene which is located within the class III region of the major histocompatibility complex between HLA-B and HLA-DR on chromosome six of the human genome. However, the above definition shall not apply to the term “tumor necrosis factor alpha,” as used herein, when the language indicates that the subject may be a member of a larger group, such as mammals. When the term TNFα is used in such a manner, the term shall mean the TNFα gene of an individual mammalian subject. For example, when a biological sample comes from a horse, then TNFα is referring to the TNFα gene of the horse. Another example, when a biological sample comes from a dog, then TNFα is referring to the TNFα gene of the dog.

As used herein, “TNF1 polymorphism” means the wild type nucleic acid sequence for the TNFα gene.

As used herein, “TNF2 polymorphism” means a mutation to the wild type nucleic acid sequence for the TNFα gene such that an adenosine is substituted at position −308.

As used herein, “hybridization” means the formation of double-helical segments through hydrogen bonding between complementary base pairs on polynucleotide strands.

As used herein, “colorimetric assay” means an assay in which a change of color indicates the presence and/or quantity of an indicator being assayed for.

As used herein, “chronic obstructive pulmonary disease” means progressive airflow limitation, mostly irreversible. This includes patients with obstructive chronic bronchitis and/or pulmonary emphysema. Patients with moderate or severe persistent asthma may also have an irreversible component of airflow limitation, and thus they may also be included in the definition of chronic obstructive pulmonary disease.

The method and kit disclosed herein are related identifying the genotype of a subject so that a treatment regimen may be developed to more effectively treat the following disease conditions. This list is for illustration, and not limitation: septic shock, rheumatoid arthritis, multiple sclerosis, hepatitis B, hepatitis C, chronic obstructive pulmonary disease, and certain cancers.

The method disclosed herein, as well as the polymorphism kit disclosed herein, requires a biological sample from a subject. Molecular techniques known in the art are used to extract nucleic acids, which include the promoter sequence of the TNFα gene. In certain embodiments, one of ordinary skill in the art may use known techniques for isolating the nucleic acid from a biological sample. The total nucleic acid which is isolated from a biological sample will include tumor necrosis factor alpha. The genomic DNA for tumor necrosis factor alpha comprises a promoter region, introns and exons.

The well known molecular techniques, known to those of ordinary skill in the art, provide a sufficient amount of the promoter of the TNFα gene such that polymerase chain reaction may be used to amplify the nucleic acid.

The methods disclosed herein include the use of the polymerase chain reaction. More specifically, once nucleic acids are extracted from a clinical specimen, specific primers which are designed to allow amplification of the organism DNA by PCR are used. In certain embodiments, the forward PCR primer has the sequence: GGCCACTGACTGATTTGTGTGTA and the reverse PCR primer has the sequence: AAGAAATGGAGGCAATAGGTTTTG. In certain embodiments, Taq DNA polymerase incorporates digoxigenin-11-dUTP (DIG) into the amplification product. As one of ordinary skill in the art knows, forward and reverse primers having sequences that are complementary to the known sequence of TNFα will suffice as PCR primers as long as they flank the region of the nucleotide sequence which is to be amplified.

Polymerase Chain Reaction

As disclosed in this invention, the polymerase chain reaction (PCR) is used to amplify the region of nucleic acid sequence which contains the specific base or bases of a polymorphism. In certain embodiments, a region of the promoter of the TNFα gene containing nucleotide −308 is amplified by PCR. Generally, conditions for PCR are well known by those of ordinary skill in the art. More specifically, PCR conditions that are useful to the invention disclosed herein are provided in the art. (Tang, et al., J. Clinical Microbiology (1998) 36:2714-2717). Specific examples providing the actual conditions used in the method of the present invention are disclosed herein.

Probe Biotinylation

Biotinylation of the hybridization probes described within the disclosed invention is accomplished by a commercial source. Integrated DNA Technologies, Inc. (Coralville, Iowa) synthesizes primers and probes upon request. Integrated DNA Technologies, Inc. also performs the 5′-biotinylation. In certain embodiments, a probe may have a minor groove binder to allow the hybridization of a shorter probe (7-15 bases) so that the presence or absence of a single nucleotide will have a greater weight in determining whether the probe hybridizes to the target sequence. Minor groove binders are components that bind to the minor groove of the double helix. The presence of a minor groove binder allows hybridization to occur with shorter than usual oligonucleotide sequences. Examples of minor groove binders include, but are not limited to, 1,2-dihydro-(3H)-pyrrolo[3,2-e]indole-7-carboxylate (CDPI₃), other compositions known to those of ordinary skill in the art, and the minor groove binders specifically mentioned in the following articles, which are incorporated herein by reference. (Afonina, et al., Nucleic Acids Research (1997) 25:2657-60; Kutyavin, et al., Nucleic Acids Research (1997) 25:3718-23). As discussed in the example section of this application, 5′ biotin-labeled probes, having minor groove binders, are commercially available.

Biotin-labeled probes are used to hybridize to the amplification products. In certain embodiments, the first biotin-labeled probe has the following nucleic acid sequence: ACCCCGTCCcCATG, and the second biotin-labeled probe has the following sequence: ACCCCGTCCtCATG. In other embodiments, other biotin-labeled probes may be used as long as the probes have nucleotide sequences that are complementary to the TNFα sequence and the probes are long enough to allow hybridization. In other certain embodiments, a hybridization probe may be a portion of either SEQ ID NO:1 or SEQ ID NO: 2.

The function of the biotin-labeled probe is to allow an interaction with a streptavidin-coated surface. In certain embodiments, the biotin-labeled probes may interact with a streptavidin-coated surface, such as a 96 well microtiter plate. In other embodiments, a streptavidin-coated reaction chamber may be used to allow the immobilization of the biotin-labeled probe. Compositions that immobilize the probe may be used. In other embodiments, the hybridization probes may be labeled with other components known to those of ordinary skill in the art which would allow the capture, or immobilization, of the probe-target complex during subsequence steps of the method disclosed herein. In certain embodiments, once the probe hybridizes, the resulting complexes are captured on streptavidin-coated microtiter plate wells.

Hybridization of Amplification Product

Subsequent to the PCR reaction, the amplification product is mixed with a hybridization solution having a biotin-labeled probe. The amplification product is split such that independent and separate hybridization reactions involving independent and separate biotin-labeled probes may occur.

The present invention discloses a method of detecting a single nucleotide polymorphism (SNP). Single nucleotide polymorphisms are polymorphisms involving only a single nucleotide. Accordingly, detection of a SNP involves detecting the identity of the single nucleotide. The method disclosed herein comprises amplifying the nucleotide sequence containing the SNP and hybridizing a probe to the amplification product in order to capture or immobilize the amplification product. In certain embodiments, two aliquots of denatured PCR amplification products are mixed with two hybridization solutions. One hybridization solution contains a 5′ biotin-labeled oligonucleotide probe having the sequence of SEQ ID NO. 1 (for TNF1), or portion thereof. The second hybridization solution contains a 5′ biotin-labeled oligonucleotide probe having the sequence of SEQ ID NO. 2 (for TNF2), or portion thereof. In certain embodiments, the hybridization conditions include incubation for from about 15 minutes to about 2 hours, preferably about one hour, in a hybridization buffer, having a final concentration of 5× Denhardt's solution, 5× SSPE, 0.5% SDS, and 100 μg/ml denatured salmon sperm DNA. Specific examples of the hybridization conditions are disclosed herein.

Detection of Color Change

Once the amplification product-probe complex is captured or immobilized, it is necessary to detect its presence. In certain embodiments, the presence of the amplification product-probe complex is preferably detected with a color change by using peroxidase-conjugated anti-DIG antibody. In other embodiments, one of ordinary skill in the art may use the following to detect the presence of the DIG labeled amplification product: alkaline phosphatase, rather than peroxidase. Additionally, detection may be accomplished by using chemoluminescent materials rather than the above-mentioned materials.

If proper target DNA is present, the biotin-labeled probe hybridizes to form an amplification product-probe complex. The amplification product-probe complex is captured on a streptavidin-coated surface. Such complexes are detected by using an anti-digoxigenin-peroxidase conjugate that attaches to the digoxigenin-11-dUTP substitutions located within the nucleic acids generated during the PCR protocol described herein. Color development occurs when a peroxidase substrate is mixed with the anti-digoxigenin-peroxidase conjugate. Peroxidase substrates are known to those of ordinary skill in the art. An example of a peroxidase substrate is 2,2′-azino-di-3-ethylbenzthiazoline sulfanate (ABTS) (Roche Diagnostics, Indianapolis, Ind.).

Color development is measured by an optical density reading. One of ordinary skill in the art is familiar with the protocols used to obtain optical density (OD) readings for samples contained in a microtiter plate. Many instruments capable of performing the measurement of optical density are commercially available. Optical density of the solution is measured at a single wavelength of about OD₄₀₅ or about OD₄₉₀. Preferably, optical density of the solution is measured at dual wavelengths, of about OD₄₀₅ and about OD₄₉₀. A blank is commonly used to correct the baseline of the instrument in order to assure accurate readings.

The methods disclosed herein provide clear results. The positive versus negative (P/N) ratio for this method is about 1000. Stated another way, the OD value of a positive result is about 1000 times greater than the OD value of a negative result. Again, a positive result indicates that target DNA that hybridizes to the 5′ biotin-labeled probe is present in the amplification product. Conversely, a negative result indicates that target DNA that hybridizes to the 5′ biotin-labeled probe is not present in the amplification product.

Polymorphism Kit

The present invention additionally discloses a polymorphism kit. The kit includes the components needed to practice the disclosed methods of identifying a nucleotide variation in the sequence of a nucleic acid and identifying a genetic predisposition in a mammal for nonresponsiveness to either Etanercept (Enbrel, Immunex, Seattle, Wash.) or Infliximab (Remicade, Centocor, Malvern, Pa.) treatment.

Upon detection of the TNF2 polymorphism, the patient should consider specific drug treatment. TNFα production may be inhibited by using drugs known by those of ordinary skill in the art to have such effects. In certain embodiments, Etanercept (Enbrel, Immunex, Seattle, Wash.), a soluble TNFα receptor fusion protein, may be used to bind and inactivate TNF. Accordingly, Etanercept has been used to treat adults and children. (Lovell, et al., New England J. Med. (2000) 342:763-769; Moreland, et al., New England J. Med. (1997) 337:141-7; Weinblatt, et al., New England J. Med. (1999) 340:253-9; Moreland, et al., Ann. Intern. Med. (1999) 130:478-86; Bathon, et al., New England J. Med. (2000) 343:1586-1593). In alternate embodiments, Infliximab (Remicade, Centocor, Malvern, Pa.) is used to treat the TNF2 polymorphism. Infliximab is a chimeric monoclonal antibody against TNFα. The drug provides a clinical benefit when administered alone or in combination with methotrexate. (Maini, et al., Arthritis Rheum. (1998) 41:1552-63; Kavanaugh, et al., J. Rheumatol. (2000) 27:841-50; Maini, et al., Lancet (1999) 354:1932-9; Lipsky, et al., New England J. Med. (2000) 343:1594-1602). Infliximab has been shown to significantly reduce the signs and symptoms of rheumatoid arthritis. (Elliot et al., Arthritis Rheum., 1993, 35:1681-1690). Infliximab is a chimeric monoclonal antibody, having an approximate molecular weight of 149,100 daltons, that is sufficient to neutralize human TNFα. (Maini et al., Annu. Rev. Med., 2000, 51:207-229).

The practice of the method described herein may be easily accomplished by use of a prefabricated kit that includes either a portion or all of the materials needed to practice the method disclosed herein. In certain embodiments a polymorphism kit includes only materials that are not commonly found in a standard molecular biology laboratory or a clinical diagnostic laboratory. By way of example, but not limitation, such a kit preferably includes PCR forward primer, PCR reverse primer, first biotinylated probe having a minor groove binding component, and second biotinylated probe having a minor groove binding component. In other embodiments, a polymorphism kit includes the PCR primers used to amplify the promoter of the TNFα gene. In other embodiments, the polymorphism kit includes PCR primers, biotin-labeled hybridization probes having a minor groove binder, instructions regarding the method disclosed herein, streptavidin-coated microtiter plates and peroxidase conjugated anti-DIG antibody. It may be desirable for a variety of kits to be offered wherein each kit contains either the minimal supplies, for example, PCR primers and hybridization probes, or almost all materials necessary to practice the method disclosed herein.

### EXAMPLE 1

Amplification of the Nucleic Acid Obtained from a Biological Sample

Following the isolation of genomic DNA from a biological sample, PCR is used to amplify a segment of the DNA. The following primers are hybridized to the template: SEQ ID NO: 3 and SEQ ID NO: 4. The PCR mixture contains the following: 1×EN buffer, 18% glycerol, 300 uM dATP, dCTP, and dGTP, 285 uM dUTP, 15 uM digoxigenin-11-dUTP (Roche Diagnostics, Inianapolis, Ind.), 0.5 uM of each primer and Taq polymerase. The final reaction volume of the PCR reaction is 50 microliters (ul). The reaction mixtures were placed in a Perkin-Elmer 9600 thermal cycler programmed for a three-step PCR procedure. For additional details regarding PCR processes see Tang et al., J. Clin. Microbiol. (1998) 36:2714-2717; Tang et al., Dign. Microbiol. Infect. Dis. (1999) 34:333-337. Subsequent to the PCR procedure, 20 ul of the PCR reaction volume are mixed with 5 ul of denaturing buffer (eg. 10% NaOH). The mixture is incubated at room temperature for 10 minutes.

### EXAMPLE 2

Hybridization of Biotin-Labeled Probe to Amplification Product

Oligonucleotide probes having 5′-biotinylation and minor groove binder modification are commercially available from Applied Biosystems, Foster City, Calif. 94404. The final concentration of the biotin-labeled probe used during the hybridization reaction is from about 0.5 (micromolar) uM to 5 uM. Preferably, the final concentration of the biotin-labeled probe used during the hybridization reaction is 1 uM. The biotin-labeled probe has the sequence of SEQ ID NO: 1. Probe hybridization buffer includes 5× Denhardt's solution, 5× SSPE, 0.5% SDS, and 100 μg/ml denatured salmon sperm DNA.

A single well of a microtiter plate is used as an incubation chamber. To the incubation chamber, the following components are added: 175 ul of probe hybridization buffer, 25 ul of denatured PCR product (the 20 ul of PCR reaction volume with 5 ul of denaturing buffer, disclosed in example 1), and an amount of the biotin-labeled probe such that the final concentration of the biotin-labeled probe in the hybridization reaction volume is 1 uM. Accordingly, the final reaction volume is 200 ul. Hybridization occurs at 37° C. for one hour, while shaking. For additional details regarding the use of a minor groove binder see Kutyavin et al., Nucleic Acids Research, 1997, 25:3718-3723; Afonina et al., Nucleic Acids Research, 1997, 25:2657-2660.

### EXAMPLE 3

Capture of Biotin-Labeled Probe and Amplification Product Binding Thereto

The above-mentioned biotin-labeled probe is captured by incubating the probe in a streptavidin-coated microtiter plate for a period of one hour at 37° C. Since the microtiter plate used in example 2 was coated with streptavidin, this step was accomplished in example 2. Subsequent to incubation, which allows hybridization of the biotin-labeled probe to the amplification product and attachment of the biotin-labeled probe to streptavidin, the well is washed 5 times with wash buffer. The wash buffer is 1×PBS and 0.01% tween-80.

### EXAMPLE 4

Detection of Amplification Product with Peroxidase

Following hybridization of the amplification product to the biotin-labeled probe and capture of the amplification product-probe complexes, the presence of the digoxigenin-11-dUTP containing amplification product is determined. The presence of digoxigenin-11-dUTP is detected by adding peroxidase-conjugated anti-DIG antibody. The peroxidase-conjugated anti-DIG antibody is commercially available from Roche Diagnostics (Indianapolis, Ind.). The manufacturer's recommendations are followed with regard to usage. The final volume in which the peroxidase-conjugated anti-DIG is added is 200 ul. Accordingly, a volume of 200 ul is added to the incubation chamber. The incubation chamber is covered and incubated at 37° C. for 30 minutes. Subsequent to incubation, the incubation chamber is washed five times with wash buffer. The wash buffer has a final concentration of 1×PBS and 0.01% tween-80.

After washing, 2,2′-azino-di-3-ethylbenzthiazoline sulfanate (ABTS) (Roche Diagnostics, Indianapolis, Ind.) is added. ABTS is a chromogenic substrate for peroxidase. ABTS, in the form of a single tablet (Roche Diagnostics, Indianapolis, Ind.), is dissolved in 10 milliliters (ml) of water to produce an ABTS solution. 200 ul of the ABTS solution is added to the incubation chamber. The incubation chamber is covered and incubated for 30 minutes at 37° C., while shaking.

Color development is measured by an optical density reading. One of ordinary skill in the art is familiar with the protocols used to obtain optical density (OD) readings for samples contained in a microtiter plate. Optical density of the solution is measured at dual wavelengths, OD₄₀₅ and OD₄₉₀. While measuring the optical density, a blank is used to correct the baseline. The positive versus negative (P/N) ratio for this method is about 1000.

What is claimed is:

1. A method of identifying a nucleotide variation in sequence in a nucleic acid, comprising:
providing a biological sample having a nucleic acid;
contacting the nucleic acid with a primer under conditions for nucleic acid amplification, wherein the primer amplifies the nucleic acid in order to produce an amplification product, wherein the nucleic acid is amplified by polymerase chain reaction;
contacting a first aliquot of the amplification product with a first solution having a first biotin-labeled probe, the first biotin-labeled probe having a minor groove binder so that an amplification product-probe complex is formed; and
detecting the amplification product-probe complex.

2. The method of claim 1, further comprising isolating the nucleic acid from the biological sample, wherein the nucleic acid is a Tumor Necrosis Factor α gene having a promoter, wherein the promoter is amplified by polymerase chain reaction from the nucleic acid isolated from the biological sample.

3. The method of claim 2, wherein digoxigenin-11-dUTP is incorporated into the amplification product.

4. The method of claim 3, further comprising incubating the first solution on a first streptavidin coated surface so that the first biotin-labeled probe is fixed to the first streptavidin coated surface.

5. The method of claim 4, further comprising contacting a second aliquot of the amplification product with a second solution having a second biotin-labeled probe, the second biotin-labeled probe having the minor groove binder, wherein the second biotin-labeled probe hybridizes to a TNF2 polymorphism.

6. The method of claim 5, further comprising incubating the second solution on a second streptavidin coated surface so that the second biotin-labeled probe is fixed to the second streptavidin coated surface.

7. The method of claim 6, further comprising detecting the digoxigenin-11-dUTP on the second streptavidin coated surface by peroxidase-conjugated anti-digoxigenin-11-dUTP antibody.

8. The method of claim 7, wherein the second biotin-labeled probe further comprises a nucleic acid sequence having a length from about 7 nucleotides to about 14 nucleotides, having the sequence of SEQ ID NO: 2.

9. The method of claim 7, wherein the second biotin-labeled probe further comprises SEQ ID NO: 2.

10. The method of claim 4, wherein contacting the nucleic acid with the primer under conditions for nucleic acid amplification further comprises contacting under conditions suitable for hybridization between complementary nucleic acid sequences.

11. The method of claim 10, wherein the first biotin-labeled probe further comprises a nucleic acid sequence having a length from about 7 nucleotides to about 14 nucleotides, having the sequence of SEQ ID NO: 1.

12. The method of claim 10, wherein the first biotin-labeled probe further comprises SEQ ID NO: 1.

13. A method of identifying a genetic predisposition in a mammal, comprising:
providing a biological sample having genomic DNA from the mammal, wherein the genomic DNA is tumor necrosis factor alpha;
amplifying the genomic DNA in order to produce an amplification product;
providing a hybridization solution so that a probe hybridizes to the amplification product;
hybridizing the probe to the amplification product so that an amplification product-probe complex is formed;
immobilizing the probe; and
determining the presence of the amplification product-probe complex, wherein the presence of the amplification product-probe complex identifies a single nucleotide polymorphism in the tumor necrosis factor alpha genomic DNA.

14. The method of claim 13, wherein the probe further comprises SEQ ID NO: 1.

15. The method of claim 13, wherein the probe further comprises SEQ ID NO: 2.

16. The method of claim 13, wherein the probe is selected from a group consisting of SEQ ID NO: 1, SEQ ID NO: 2, complement of SEQ ID NO: 1, and complement of SEQ ID NO: 2.

17. The method of claim 13, wherein the biological sample is a blood sample.

18. The method of claim 13, wherein the mammal is a rheumatoid arthritis patient.

19. The method of claim 13, wherein the mammal is a septic shock patient.

20. The method of claim 13, wherein the mammal is a multiple sclerosis patient.

21. The method of claim 13, wherein the mammal is a hepatitis B patient.

22. The method of claim 13, wherein the mammal is a hepatitis C patient.

23. The method of claim 13, wherein the mammal has a chronic obstructive pulmonary disease.

24. A diagnostic kit for detecting single nucleotide polymorphisms in mammals, comprising:
a forward polymerase chain reaction primer;
a reverse polymerase chain reaction primer;
a first probe having a first minor groove binding component;
a second probe having a second minor groove binding component; and
a set of instructions for use.

25. The kit of claim 24, wherein the forward polymerase chain reaction primer and reverse polymerase chain reaction primer have a length from about 15 to about 100 nucleotides in length, wherein the forward polymerase chain reaction primer and reverse polymerase chain reaction primer further comprise nucleic acid sequence that is complementary to nucleic acid sequence of a tumor necrosis factor alpha promoter.

26. The kit of claim 25, wherein the first probe further comprises SEQ ID NO: 1.

27. The kit of claim 26, wherein the second probe further comprises SEQ ID NO: 2.

28. The kit of claim 25, wherein the diagnostic kit further comprises digoxigenin-11-dUTP.

29. The kit of claim 28, wherein the diagnostic kit further comprises a streptavidin coated surface.

30. The kit of claim 29, wherein the diagnostic kit further comprises a peroxidase-conjugated anti-digoxigenin-11-dUTP antibody.
